(19) United States
(12) Patent Application Publication
(10) Pub. No.: US 2025/0186759 A1
(43) Pub. Date: Jun. 12, 2025

(54) CIRCULATORY SUPPORT DEVICES, SYSTEMS, AND METHODS

(71) Applicant: Boston Scientific Scimed, Inc., Maple Grove, MN (US)

(72) Inventors: Judith Tiferes Wang, Shoreview, MN (US); Jennifer Gibbs, White Bear Lake, MN (US); Alan Torborg, Minneapolis, MN (US)

(73) Assignee: Boston Scientific Scimed, Inc., Maple Grove, MN (US)

(21) Appl. No.: 18/972,464

(22) Filed: Dec. 6, 2024

Related U.S. Application Data
(60) Provisional application No. 63/607,788, filed on Dec. 8, 2023.

(51) Int. Cl. A61M 60/585; A61M 60/122; A61M 60/592

(52) U.S. Cl. CPC A61M 60/585; A61M 60/122; A61M 60/592; A61M 2205/04; A61M 2205/10; A61M 2205/3553; A61M 2205/3561; A61M 2205/3592; A61M 2205/502; A61M 2205/52

(57) ABSTRACT

A circulatory support system may include a blood pump, a console in communication with the blood pump, a first user interface, and a second user interface. The console may include a controller configured to control operation of the blood pump. The first user interface may be configured to display a live view related to operation of the blood pump. The first user interface may be part of the console. The second user interface may be configured to display live data and historical data of the one more parameters related to operation of the blood pump. The second user interface may be remote from the console. A screen displaying the live view on the first user interface may have a predetermined configuration. A screen displaying the live view and the historical data on the second user interface may be configured to be adjusted over time.

### CROSS-REFERENCE TO RELATED APPLICATIONS

This application claims the benefit of priority under 35 U.S.C. § 119 of U.S. Provisional Application No. 63/607,788, filed Dec. 8, 2023, the entire disclosure of which is hereby incorporated by reference.

### TECHNICAL FIELD

The present disclosure pertains to mechanical circulatory support devices, systems, and methods. More specifically, the present disclosure relates to percutaneous ventricular assist device (PVAD) systems with user interfaces and displays.

### BACKGROUND

A wide variety of intracorporeal and extracorporeal medical devices and systems have been developed for medical use, for example, in cardiac procedures and/or for cardiac treatments. Some of these devices and systems include guidewires, catheters, catheter systems, pump devices, circulatory assist devices, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including ventricular assist devices.

In a first example, a circulatory support system may comprise a blood pump, a console in communication with the blood pump, the console comprising a controller configured to control operation of the blood pump, a first user interface configured to display a live view related to operation of the blood pump, the first user interface may be part of the console, and a second user interface configured to display live data and historical data of one or more parameters related to operation of the blood pump, the second user interface is remote from the console.

Alternatively or additionally to any of the examples above, in another example, the first user interface may be configured to display a first screen including the live view related to operation of the blood pump and a second screen configured to facilitate a user controlling operation of the blood pump.

Alternatively or additionally to any of the examples above, in another example, the first screen and the second screen may have preconfigured fixed layouts and may be the only screens that the console is configured to display.

Alternatively or additionally to any of the examples above, in another example, the second user interface may be a dynamic user interface configured to adjust between a plurality of screens based on user interaction with a screen of the plurality of screens displayed on the second user interface.

Alternatively or additionally to any of the examples above, in another example, the plurality of screens may be configured to display one or more of a live view of one or more patient parameters related to operation of the blood pump, a historical view of one or more patient parameters related to operation of the blood pump, operational settings of the blood pump, and a view of one or more patient parameters related to operation of the blood pump for a plurality of patients.

Alternatively or additionally to any of the examples above, in another example, the second user interface may include one or more selectable elements associated with data received from the console and the one or more selectable elements are configured to be selected to cause the second user interface to display a screen with additional information related to the data received from the console.

Alternatively or additionally to any of the examples above, in another example, the one or more selectable elements may be a patient pane on a screen displaying a plurality of patient panes and the screen with additional information related to the data received from the console includes a plurality of adjustable patient panes that include one or more of a live view of one or more patient parameters, historical alarm data, and historical waveform data.

Alternatively or additionally to any of the examples above, in another example, the first user interface may include a first display having a first surface area and the second user interface may include a second display having a second surface area greater than the first surface area.

Alternatively or additionally to any of the examples above, in another example, the second user interface may be configured to receive one or both of the live data and the historical data from the console over a local area network.

Alternatively or additionally to any of the examples above, in another example, the second user interface may be configured to receive one or both of the live data and the historical data from the console over a wide area network.

Alternatively or additionally to any of the examples above, in another example, the console may be configured to store the live data and the historical data of one or more parameters related to operation of the blood pump.

Alternatively or additionally to any of the examples above, in another example, the console may be configured to output the live data and the historical data of one or more parameters related to operation of the blood pump to a server in communication with a computing device comprising the second user interface.

In another example, a console for a circulatory support system may comprise a controller configured to control operation of a blood pump of the circulatory support system, a first display configured to display a live view of one or more parameters related to operation of the blood pump, and wherein the controller may be configured to store and output live data and historical data of one or more parameters related to operation of the blood pump.

Alternatively or additionally to any of the examples above, in another example, the controller may be configured to communicate with the blood pump over a wired connection.

Alternatively or additionally to any of the examples above, in another example, the controller may be configured to output the live data and the historical data of one or more parameters related to operation of the blood pump to a remote user interface over a wired connection.

Alternatively or additionally to any of the examples above, in another example, the controller may be configured to output the live data and the historical data of one or more parameters related to operation of the blood pump to a remote user interface over a wireless connection.

In a further example, a method may comprise receiving data over time at a console from a blood pump operating within a patient, wherein the data received includes data related to operation of the blood pump within the patient, displaying live data from the data received over time on a first user interface of the console, communicating the data received over time from the console to a remote computing device having a second user interface, and displaying data related to operation of the blood pump on the second user interface.

Alternatively or additionally to any of the examples above, in another example, the method may further comprise storing the data received over time on the console.

Alternatively or additionally to any of the examples above, in another example, the method may further comprise displaying the live data and historical data on a third user interface remote from the first user interface and the second user interface.

Alternatively or additionally to any of the examples above, in another example, the method may further comprise receiving a selection of a selectable element on a screen of the second user interface, wherein the screen is displaying at least some of the data related to operation of the blood pump, and in response to receiving the selection of the selectable element, displaying a detailed view that includes data associated with the selectable element.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic diagram of an illustrative circulatory support system with a blood pump positioned in a patient;

FIG. 2 is a schematic diagram of an illustrative computing system;

FIG. 3 is a schematic diagram of an illustrative circulatory support system that includes a remote display;

FIG. 4 is a schematic diagram of an illustrative circulatory support system with a blood pump that includes a remote display;

FIG. 5 is a schematic diagram of an illustrative circulatory support system that includes a remote display and a plurality of circulatory support devices;

FIG. 6 is a schematic diagram of an illustrative circulatory support system that includes a plurality of remote displays;

FIG. 7 is a schematic diagram of an illustrative circulatory support system that includes a plurality of remote displays;

FIG. 8 is a schematic configuration of an illustrative screen of a circulatory support system;

FIG. 9 is a schematic configuration of an illustrative screen of a circulatory support system;

FIG. 10 is a schematic configuration of an illustrative screen of a circulatory support system;

FIG. 11 is a schematic configuration of an illustrative screen of a circulatory support system;

FIG. 12 is a schematic configuration of an illustrative screen of a circulatory support system;

FIG. 13 is a schematic configuration of an illustrative screen of a circulatory support system;

FIG. 14 is a schematic configuration of an illustrative screen of a circulatory support system; and

FIG. 15 is a schematic configuration of an illustrative screen of a circulatory support system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an configuration”, “some configurations”, “other configurations”, etc., indicate that the configuration described may include one or more particular features, structures, and/or characteristics. However, such recitations do not necessarily mean that all embodiments include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

A variety of circulatory support devices and systems are known for assisting or replacing a pumping function of a heart in a patient with severe heart failure and/or other cardiac conditions. Circulatory support devices may be configured to treat patients with cardiogenic shock, myocardial infarction, acutely decompensated heart failure, and/or other heart related conditions. Additionally or alternatively circulatory support devices may support a patient during percutaneous coronary interventions and/or other procedures.

Example cardiac circulatory support devices include, but are not limited to, ventricular assist devices (VADs), total artificial hearts, intra-aortic balloon pumps (IABP), and extracorporeal membrane oxygenation (ECMO). Example VADs include left ventricular assist devices (LVADs), right ventricular assist devices (RVADs), and biventricular assist devices (BiVADs). A further illustrative VAD is a percutaneous ventricular assist device (PVAD), which may be inserted into a ventricle (e.g., a left ventricle or a right ventricle) of a heart of a patient via delivery through a femoral artery or vein and/or other suitable vasculature to the ventricle. As a PVAD may be placed at a desired location of anatomy of a patient via percutaneous access and delivery, the PVAD may be used in emergency medicine, a catheter laboratory, and/or other surgical and/or non-surgical settings.

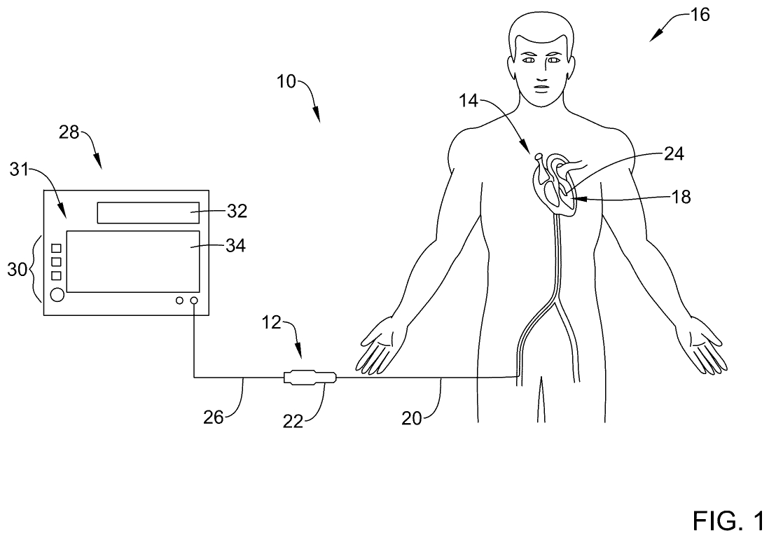

FIG. 1 depicts an illustrative percutaneous circulatory support system 10 including a circulatory support device 12 positioned in the heart 14 of a patient 16. The circulatory support device 12 may include, among other components, a flexible elongate catheter shaft 20 having a first end attached to a handle 22 and a second end attached to a blood pump 24. As depicted in FIG. 1, the blood pump 24 may be positioned in the left ventricle 18 of the patient 16. The blood pump 24 may be delivered (e.g., tracked) to the left ventricle 18 percutaneously over a guidewire. For example, the catheter shaft 20 and the blood pump 24 may be tracked over a guidewire through the femoral artery and the descending aorta, over the aortic arch, through the ascending aorta, past the aortic valve, and into the left ventricle 18.

In some examples, the blood pump 24 may be positioned within the heart 14 such that one or more blood inlets positioned along the distal region of the blood pump 24 may be located in the left ventricle 18 and one or more blood outlets positioned along a housing of the blood pump 24 may be located in the aorta. Additionally, the blood pump 24 may include an electrically powered motor that drives rotation of an impeller (e.g., where the motor and/or the impeller may be positioned within the housing of the blood pump 24). In some examples, the motor may power the rotation of the impeller via electromagnetic induction, but other suitable configurations are contemplated. The rotating impeller may draw blood from the left ventricle (via the one or more of the blood inlets) into the aorta (via the one or more blood outlets). In other words, an electrically powered motor may drive the impeller to pump blood from the left ventricle through the aortic valve and into the ascending aorta.

FIG. 1 further illustrates that a first end of the catheter shaft 20 may be attached to a handle 22. The handle 22 may include a distal end region attached to the catheter shaft 20 and a proximal end region attached to a cable 26. It can be appreciated that the handle 22 may include one or more actuators (e.g., buttons, levers, dials, switches, etc.) designed to permit a clinician to control various functions of the blood pump 24. For example, a clinician may be able to control the speed of the motor and/or the impeller located in the blood pump 24 via actuation of one or more actuators located on the handle 22.

The cable 26 may include a proximal end region connected to a console 28. The cable 26 may include or be an electrical power cable configured to power the blood pump 24, a communication cable configured to communicate data between the blood pump 24 and the console 28, an electrical cable, an optical cable, and/or other suitable type of cable.

The percutaneous circulatory support system 10 may include one or more sensors positioned within and/or along the console 28, the handle 22, the catheter shaft 20, and/or the blood pump 24, where the sensors may be coupled with the console 28 and/or other data monitoring component via wired connection (e.g., an electrical and/or optical connection of the cable 26) and/or a wireless connection. The one or more sensors positioned within and/or along the console 28, within the handle 22, the catheter shaft 20, and/or the blood pump 24 may be configured to sense one or more parameters of or related to operation of the circulatory support system 10, operation of the circulatory support device 12, and/or the patient 16. The one or more sensors positioned within and/or along the console 28, the handle 22, along the catheter shaft 20, and/or the blood pump 24 may be designed or configured to sense and/or monitor any suitable parameters including, but not limited to, blood pressures (e.g., arterial pressure, venous pressure), blood velocity, blood flowrate, mean arterial pressure, impeller speeds, motor speeds, an electrical current provided to the motor, voltage provided to the motor, back-EMF from the motor, and/or other suitable parameters, along with any suitable combination or temporal pattern of signals corresponding to one or more of the parameters. Further, additional parameters (e.g., flow through or across the blood pump 24) may be derived by processing combinations of sensed data in a time dependent manner.

As depicted in FIG. 1, the console 28 may include one or more control knobs (e.g., buttons, knobs, dials, touch-sensitive areas, etc.) 30 and/or one or more displays 31. FIG. 1 depicts the console 28 with a first display 32 and a second display 34, but other suitable configurations of the console 28 with a single display and/or with more than two displays are contemplated. Additionally, while FIG. 1 illustrates the first display 32 and the second display 34 integrated into the console 28, it is contemplated that the circulatory support system 10 may be designed such that the first display 32, the second display 34, or both of the first display 32 and the second display 34 are separate, distinct components of the circulatory support system 10. In other words, the first display 32, the second display 34, or both of the first display 32 and the second display 34 may be separate stand-alone displays, apart from the console 28 (e.g., remote displays 31). In some examples, the first display 32 and the second display 34 may receive data from the same or different sources. An example circulatory support system including a console with two displays is disclosed in Appl. No. 63/457,935, filed on Apr. 7, 2023, and titled CIRCULATORY SUPPORT DEVICE SYSTEM, which is hereby incorporated by reference in its entirety for any and all purposes.

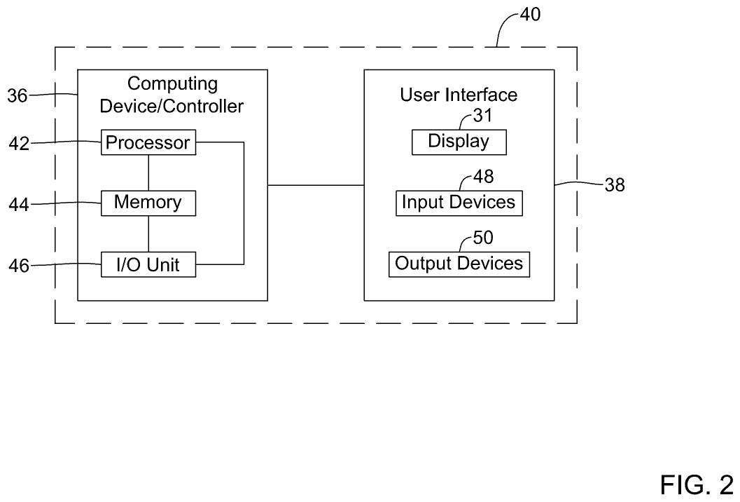

The console 28 and/or other components of the circulatory support system 10 may be or may include a computing device or controller including or coupled with a user interface. FIG. 2 depicts a schematic box diagram of an illustrative computing device or controller 36 and a user interface 38, where the computing device or controller 36 and/or the user interface 38 may be entirely or partially housed in one or more housings 40. The housing 40 may be an optional component, as represented by the broken lines defining the housing 40 depicted in FIG. 2. Although various components are depicted as being included in the computing device or controller 36 and the user interface 38, one more of the depicted components may be omitted and/or one or more additional or alternative components may be utilized.

The computing device or controller 36 may be any suitable computing device configured to process data of or for the circulatory support system 10 and may be configured to facilitate operation of the circulatory support system 10. The computing device or controller 36, in some cases, may be configured to control operation of the blood pump 24 by establishing and/or outputting control signals to the blood pump 24 and/or components of or in communication with the console 28 to control and/or monitor operation of the blood pump 24. In some examples, the controller 36 may be part of the console 28 and communicate with the blood pump 24 over a wired connection, but other suitable configurations are contemplated.

In some cases, the controller 36 may communicate with a remote server or other suitable computing device. When the controller 36, or at least a part of the controller 36, is a component separate from a structure of the console 28, the controller 36 may communicate with electronic components of the blood pump 24, the console 28, and/or other suitable components of the circulatory support system 10 over one or more wired or wireless connections or networks (e.g., LANs and/or WANs).

The illustrative controller 36 may include, among other suitable components, one or more processors 42, memory 44, and/or one or more I/O units 46. Example other suitable components of the controller 36 that are not specifically depicted in FIG. 2 may include, but are not limited to, communication components, a touch screen, selectable buttons, and/or other suitable components of a controller. As discussed above, one or more components of the controller 36 may be separate from the console 28 and/or incorporated into the components of the console 28.

The processor 42 of the controller 36 may include a single processor or more than one processor working individually or with one another. The processor 42 may be configured to receive and execute instructions, including instructions that may be loaded into the memory 44 and/or other suitable memory. Example components of the processor 42 may include, but are not limited to, central processing units, microprocessors, microcontrollers, multi-core processors, graphical processing units, digital signal processors, application specific integrated circuits (ASICs), artificial intelligence accelerators, field programmable gate arrays (FPGAs), discrete circuitry, and/or other suitable types of data processing devices.

The memory 44 of the controller 36 may include a single memory component or more than one memory component each working individually or with one another. Example types of memory 44 may include random access memory (RAM), EEPROM, flash, suitable volatile storage devices, suitable non-volatile storage devices, persistent memory (e.g., read only memory (ROM), hard drive, flash memory, optical disc memory, and/or other suitable persistent memory) and/or other suitable types of memory. The memory 44 may be or may include a non-transitory computer readable medium. The memory 44 may include instructions stored in a transitory state and/or a non-transitory state on a computer readable medium that may be executable by the processor 42 to cause the processor 42 to perform one or more of the methods and/or techniques described herein. Further, in some cases, the memory 44 and/or other suitable memory may store data received from the blood pump 24.

The I/O units 46 of the controller 36 may include a single I/O component or more than one I/O component each working individually or with one another. Example I/O units 46 may be or may include any suitable types of communication hardware and/or software including, but not limited to, communication components or ports configured to communicate with electronic components of the circulatory support system 10 and/or with other suitable computing devices or systems. Example types of I/O units 46 may include, but are not limited to, wired communication components (e.g., HDMI components, Ethernet components, VGA components, serial communication components, parallel communication components, component video ports, S-video components, composite audio/video components, DVI components, USB components, optical communication components, and/or other suitable wired communication components), wireless communication components (e.g., radio frequency (RF) components, Low-Energy BLUETOOTH protocol components, BLUETOOH protocol components, Near-Field Communication (NFC) protocol components, WI-FI protocol components, optical communication components, ZIGBEE protocol components, and/or other suitable wireless communication components), and/or other suitable I/O units 46.

The user interface 38 may be configured to communicate with the computing device or controller 36 via one or more wired or wireless connections. The user interface 38 may include one or more display devices 31, one or more input devices 48, one or more output devices 50, and/or one or more other suitable features. In some examples, the user interface 38 may be part of or may include the computing device or controller 36.

The display 31 may be any suitable display. Example suitable displays include, but are not limited to, touch screen displays, non-touch screen displays, liquid crystal display (LCD) screens, light emitting diode (LED) displays, head mounted displays, virtual reality displays, augmented reality displays, and/or other suitable display types.

The input device(s) 48 may be and/or may include any suitable components and/or features for receiving user input via the user interface 38. Example input device(s) 48 may include, but are not limited to, touch screens, keypads, mice, touch pads, microphones, selectable buttons, selectable knobs, optical inputs, cameras, gesture sensors, eye trackers, voice recognition controls (e.g., microphones coupled to appropriate natural language processing components) and/or other suitable input devices. In one example, the input devices 48 may include a touch screen that allows for setting set points and/or selecting selectable elements for additional detail concerning data or information associated with the selectable elements, but this is not required.

The output device(s) 50 may be and/or may include any suitable components and/or features for providing information and/or data to users and/or other computing components. Example output device(s) 36 include, but are not limited to, displays, speakers, vibration systems, tactile feedback systems, optical outputs, and/or other suitable output devices.

The console 28 of the percutaneous circulatory support system 10 may be configured to facilitate control of the blood pump 24 and store and/or monitor data related to operation of the blood pump 24 (e.g., data from the blood pump 24, data from control of the blood pump 24, data from sensors of and/or associated with the blood pump 24, and/or data from other suitable sources). The console 28 may receive data over time from the blood pump 24 operating within a patient, where the received data may include patient data and/or data related to operation of the blood pump within the patient.

The console 28 may be configured so as to be sized and coupled to the blood pump 24 in a manner that allows for transporting the console 28 with the patient in which the blood pump 24 has been positioned, which may include transporting the patient via an emergency medical services (EMS) (e.g., via helicopter, ambulance, etc.) and/or transporting the patient between locations within a medical facility, such as a hospital, (e.g., between a catheter laboratory, an operating room, an intensive care unit, and/or other location within the medical facility). To assess the patient and/or operation of the blood pump 24, medical professionals may need to evaluate historical patient data and/or blood pump 24 data gathered and/or saved at or by the console 28.

To facilitate transportation of the console 28 with the patient, the console 28 may be lightweight and/or compact. However, it may be desirable for the console 28 to have a large display configured to facilitate displaying patient data and/or blood pump data that facilitates medical professionals assessing the patient.

To address the competing design concerns (e.g., lightweight and/or compact versus a large display for obtaining a full understanding of a patient's condition and/or a pump condition), a console 28 may include a first display, which may be configured to display one or more screens providing limited data or information (e.g., preconfigured screens with fixed layouts), and a remote computing device may include or may be coupled with a second display (e.g., a remote display that may be separated from the first display and/or is otherwise remote from the first display), which may display screens having augmented views relative to what is depicted on the first display. Such a configuration of the displays and screens for the displays may allow for reducing a size of the console 28 relative to when two displays are required on the console 28 and/or when a large single display is included on the console 28. Although the two displays configuration is discussed herein with respect to percutaneous circulatory support systems, similar concepts may be applied to other medical systems that include a computing device with limited display space, such as medical systems configured to be transported with a patient, medical systems for use in operating rooms with limited space, and/or other suitable medical systems.

In some examples, the first display on the console 28 may provide or display a live data screen (e.g., a first screen) having a preconfigured, fixed layout that shows live or current data received from the blood pump 24 for parameters related to operation of the pump (e.g., which may be patient parameters and/or pump parameters) and a control screen (e.g., a second screen) having a preconfigured, fixed layout that facilitates a user controlling operation of the blood pump 24 and at which set points for the blood pump may be adjusted. In some cases, the live data screen and/or the control screen are the only screens that the console 28 is configured to display on the first display, but this is not required.

The second display may be a display of/or a user interface that includes or is part of a computing device that is remote from the console 28. In some cases, the remote computing device may be in communication with the console 28 via one or more networks to receive the live and/or historical data from the blood pump that is stored at the console 28 or in a database in communication with the console 28. Once received from the console 28 and/or other database, the data related to operation of the blood (e.g., live data, historical data, etc.) may be displayed on the second display.

The second display (e.g., a remote display) may be configured to display screen layouts customizable and/or adjustable by users (e.g., by medical professionals and/or other users of the second display), to provide data for multiple pumps and/or multiple patients simultaneously, to provide detailed patient and/or pump data for one or more patients that may or may not otherwise be available on the console 28, to provide historical patient and/or pump data for one or more patients, and/or provide additional and/or alternative data that may or may not be available on the first display of the console 28. The second display may be a display at a nurses station, a display on a mobile device, a display on catheter laboratory or surgical room boom display, and/or a display at one more other suitable locations that may or may not be limited by the location of the console 28 and the first display relative to the second display (e.g., limited by whether the console 28 and a computing device associated with the second display are connected a same network, etc.) As the second display may be remote from the console 28 and/or the patient, the second display and/or screens of the second display may be password protected and/or protected using biometrics or other suitable security techniques. Further, augmented screens for the second display (e.g., screens different than screens displayed on the first display) may be assigned to certain remote displays and/or certain credentials (e.g., certain biometrics, certain usernames/passwords, etc.) using unique case identifiers (e.g., a combination of pump serial number and implantation date and/or other suitable unique case identifiers).

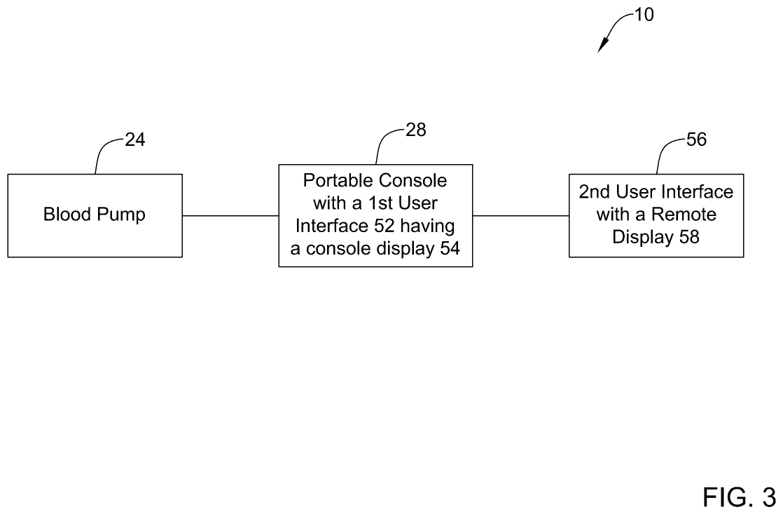

FIG. 3 depicts a schematic box diagram of an illustrative configuration of a percutaneous circulatory support system 10. The percutaneous circulatory support system 10 depicted in FIG. 3 may include, among other components, the blood pump 24, the console 28 with a first user interface 52, and a second user interface 56 in communication with the console 28. The console 28 may be considered to be a portable console 28 as it may be configured to travel with the patient as the patient moves from one location to another location.

The first user interface 52 may include a console display 54 (e.g., the first display). The console display 54 may be configured to display at least a control screen for setting setpoints of the blood pump 24 and a live view screen for viewing live patient and/or pump data. “Live data” may be data associated with a current measurement or reading from one or more sensors and/or the blood pump 24.

The console display 54 may have a first surface area with limited dimensions for displaying screens due to the portable nature of console 28. In one example, the dimensions of the console display 54 may be less than or equal to 16.5 centimeters (cm)×28 cm and/or the console display 54 may have a surface area less than 462 cm². Other suitable dimensions of the console display are contemplated.

To facilitate the first surface area with limited dimensions and/or for other purposes, screens configured for display on the console display 54 may have a preconfigured, fixed layout, may be limited in the data and/or information provided (e.g., displayed), and may provide the limited data and/or information in a clear manner that facilitates addressing current conditions of the patient and/or the blood pump 24. Example screens for display on the console display 54 are described below, but other suitable configurations of screens for display on the console display 54 are contemplated.

The second user interface 56 may be, may include, or may be part of a monitor, a mobile device, a television, a computer, and/or other suitable computing device that is remote from the console 28 (e.g., is not required to travel with the console 28 and/or is selectively directly or indirectly coupled with the console 28). The second user interface 56 may be, may include, or may be part of a medical device configured to have a proprietary wired or wireless connection with the console 28, a non-medical computing device, a low-grade medical computing device, and/or other suitable computing device.

In some configurations, the second user interface 56 may be configured to communicate with the console 28 directly through a wired or wireless connection. Additionally or alternatively, the second user interface 56 (e.g., a computing device of the second user interface 56 or a computing device encompassing the second user interface 56) may be configured to communicate with the console 28 through a server, a cloud computing system, and/or other suitable computing device(s). In some cases, the console 28 may be configured to output data and/or information related to the patient and/or operation of the blood pump 24 (e.g., live data and/or historical data of one or more parameters related to operation of the blood pump 24) to the second user interface 56, the server, a cloud computing system, and/or other suitable computing devices configured to be in communication with the console 28 over one or more networks.

When coupled with or in communication with the console 28, the second user interface 56 may be configured to receive patient and/or blood pump parameter data (e.g., live and/or historical patient and/or blood pump parameter data related to operation of the blood pump) from the console 28 for display on a remote display 58 of the second user interface 56. In some examples, the patient and/or blood pump parameter data may be provided to the second user interface 56 from the console 28 and/or other suitable computing device and/or memory component continuously, at predetermined time intervals, in response to one or more patient and/or blood pump conditions, in response to a request from the second user interface 56, and/or at one or more other suitable times.

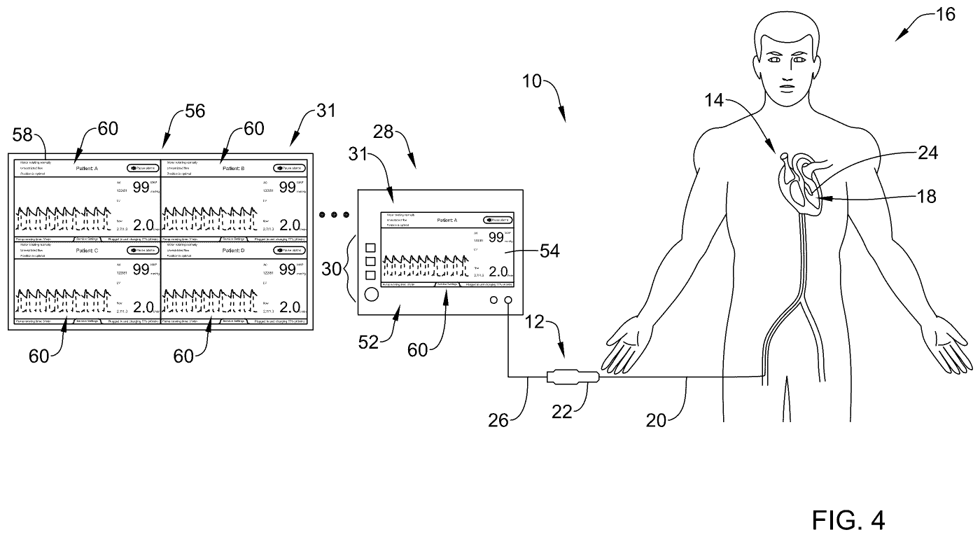

The second user interface 56 may include one or more remote displays 58 (e.g., one or more second displays). The remote display 58 may be configured to display augmented and/or adjustable screens providing live data of one or more parameters related to operation of the blood pump 24 (e.g., live patient and/or blood pump data), historical data related to operation of the blood pump 24 (e.g., historical patient and/or blood pump data), live pump and/or patient data for multiple patients simultaneously (e.g., as depicted in FIG. 4), detailed patient and/or pump data for one or more patients that is not otherwise available on the console 28, and/or provide additional and/or alternative data that may or may not be available on the first display of the console 28.

The remote display 58 may have a second surface area. Although not required, the second surface area may be greater than the first surface area of the console display 54. The larger surface area of the remote display 58 relative to the surface area of the console display 54 may allow for additional information and/or data to be provided or accessible to the user relative to the information and/or data provided on screens of the console display 54 and/or facilitate a user interacting with the information provided on screens of the remote display 58 to view additional details of patient parameters.

The second user interface 56 may be a dynamic user interface such that different screens may be displayed and/or adjusted on the remote display 58 based on (e.g., in response to) user interactions with the second user interface 56 and/or such that the data on the screens displayed on the remote display 58 is updated in real time (e.g., as the data and/or information is received). For example, when the remote display 58 may be displaying one or more of the screens of the plurality of screens (e.g., with each screen being a pane displayed), a user may select one of the displayed screens or a selectable feature within the displayed screen(s) (e.g., a user may select one or more selectable elements) and the second user interface 56 may cause one or more other screens of the plurality of screens to be displayed on the remote display 58 in addition to or as an alternative to the one or more screens that were displayed on the remote display 58 prior to the user interaction (e.g., the user selection). In some cases, the second user interface 56 may automatically rearrange, or a user may manually rearrange, the one or more screens of the plurality of screens displayed on the remote display 58 to accommodate one or more additional screens in response to the user selection and/or remove one or more of the plurality of screens displayed in response to the user selection.

FIG. 4 depicts an illustrative percutaneous circulatory support system 10 that is similar to the percutaneous circulatory support system 10 depicted in FIG. 1, but where the console 28 includes the first user interface 52 and the console display 54 (e.g., the first display) and the system 10 includes the second user interface 56 with the remote display 58 (e.g., the second display). As depicted in FIG. 4, the console display 54 may schematically depict an illustrative patient parameter live screen 60 for Patient A and the remote display 58 may schematically depict patient parameter live screens 60 for a plurality of patients (e.g., Patients A-D). Although the patient parameter live screens 60 may have an illustrative layout, set of parameters, and/or configuration, other suitable layouts, parameters, and/or configurations are contemplated. In some cases, a layout or configuration of the live screens 60 may be a predetermined or preconfigured, fixed or non-adjustable layout or configuration.

The remote display 58 of the second user interface 56 may be read-write or read-only. In some examples, the remote display 58 may be or may be part of a read-only component of the second user interface 56 or computing device in communication with the remote display 58.

As discussed, the console 28 may be configured to store data (e.g., live data, historical data, etc.) related to operation of the blood pump 24 (e.g., patient and/or blood pump data) and/or send the data related to operation of the blood pump 24 to a server (e.g., a server that is part of a LAN and/or a server that is part of a WAN). In some examples, the console 28 and/or the server may include non-volatile memory to facilitate storing data at the console 28 and/or the server and transmitting that data to remote computing devices with remote displays.

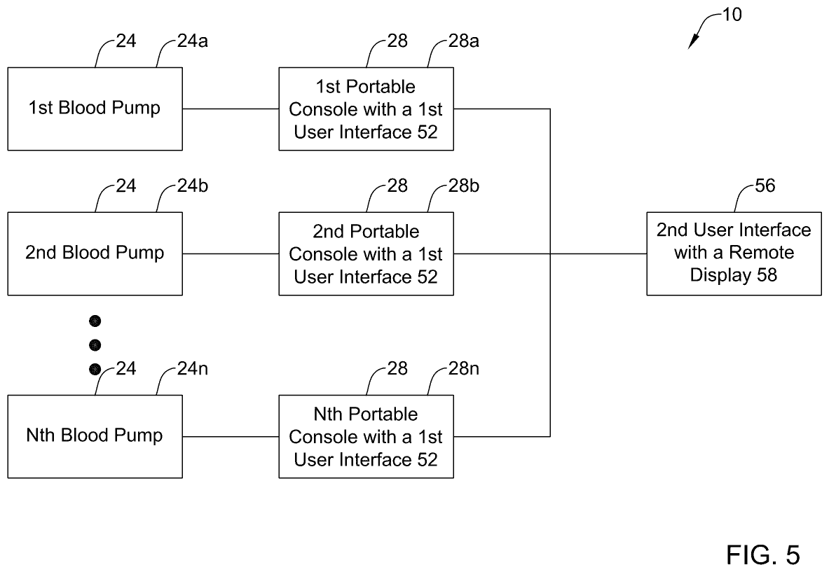

FIGS. 5-7 depict illustrative configurations of the percutaneous circulatory support system 10. Other suitable configurations of the percutaneous circulatory support system are contemplated.

FIG. 5 depicts a schematic box diagram of an illustrative configuration of the percutaneous circulatory support system 10, where the percutaneous circulatory support system 10 may include a plurality of blood pumps 24 (e.g., a first blood pump 24a, a second blood pump 24b, and an N^{th} blood pump 24n) and a plurality of consoles 28 (e.g., a first console 28a, a second console 28b, and an N^{th} console 28n) each having the first user interface 52, with each console 28 in communication with one of the plurality of blood pumps 24. In the illustrative configuration of the percutaneous circulatory support system 10 depicted in FIG. 5, the plurality of consoles 28 may be in communication with the second user interface 56 having one or more remote displays 58. When the plurality of consoles are in communication with the second user interface 56, a user may selectively choose to display different patient and/or blood pump data and/or information (e.g., live data, historical data, etc.) and/or patient and/or blood pump data and/or information for multiple patients.

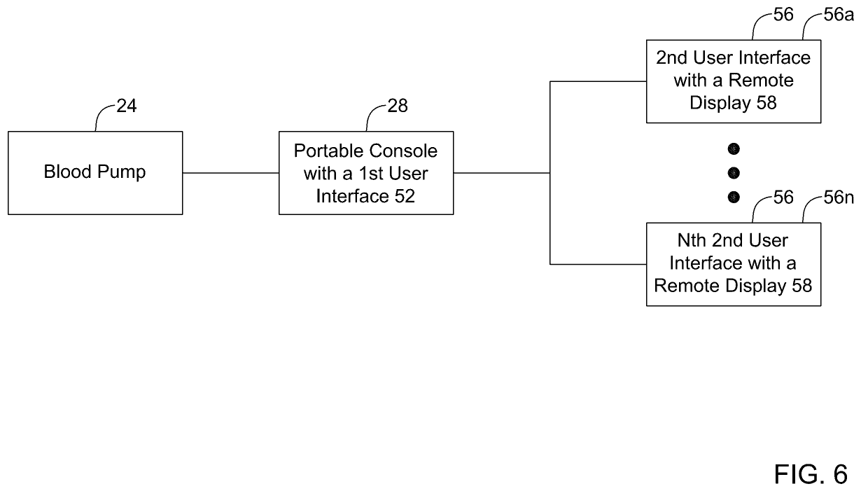

FIG. 6 depicts a schematic box diagram of an illustrative configuration of the percutaneous circulatory support system 10, wherein the percutaneous circulatory support system 10 includes one or more blood pumps 24, one or more consoles 28 with the first user interface 52, and a plurality of remote second user interfaces 56 (e.g., a first of the second user interfaces 56a and an Nth of the second user interfaces 56n), where the console 28 may be in communication with the blood pump 24 and the plurality of remote second user interfaces 56. The plurality of remote second user interfaces 56 may allow for more than one medical provider and/or other suitable user to have access to the screens of the second user interface 56 at a time (e.g., simultaneously). For example, a first user may view the screens of the second user interface 56 on a mobile device, a second user may view screens of the second user interface 56 on a display of a computing device at a nursing station, and/or a third user may view screens of the second user interface 56 on a display of a computing device in a catheter laboratory, where the screens of the second user interface 56 are related to the same blood pump 24 and/or patient. In some examples, the first user, the second user, the third user and/or other users may simultaneously view, on remote displays 58, screens of the second user interface 56 that are related to the same or different blood pump 24 and/or patient (e.g., screens displaying live and/or historical data related to operation of the blood pump 24), but this is not required. When screens of the second user interface 56 that depict data related to a single blood pump 24 and/or patient associated with the blood pump 24 are being simultaneously viewed at a plurality of remote displays 58 (e.g., at displays of a plurality of the mobile device, a computing device at a nursing station, a computing device in a catheter laboratory, etc.), the screens viewed at one of the remote displays 58 may be the same as one or more screens viewed at a different remote display 58 and/or may be different than one or more screens viewed at a different remote display 58. Although only a single blood pump 24 and a single console 28 are depicted, multiple consoles 28 coupled with blood pumps 24 may communicate with the plurality of second user interfaces 56

The screens of or configured to be viewed on the second user interface 56 may be viewed at the remote display 58 of the second user interface 56 in any suitable manner. For example, the screens may be viewed at the remote display 58 using a mobile app, a website via a web browser, a proprietary software, and/or other viewing system.

As discussed, the console 28 may be configured to communicate with the blood pump 24 and/or the second user interface(s) 56 via a wired and/or wireless connection. In some examples, the console 28 may be configured to communicate with the blood pump 24 via a wired connection, communicate with a first remote second user interface 56 via a transmission control protocol (TCP) connection or a RS232 protocol connection, and communicate with a second remote second user interface 56 via a TCP connection or a RS232 protocol connection. Additionally or alternatively, the console 28 may be configured to communicate with the blood pump 24 via a wired connection, communicate with a first remote second user interface 56 via a TCP connection or RS232 connection, and communicate with a second remote second user interface 56 via a TCP connection or WiFi connection. Other suitable configurations are contemplated.

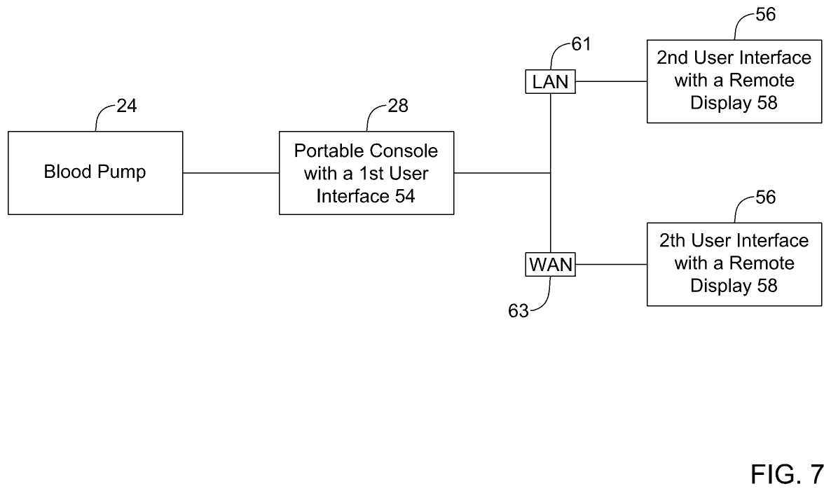

As discussed, the second user interface(s) 56 with the remote display 58 may be configured to communicate with the console(s) 28 over one or more networks. For example, the second user interface(s) 56 may be configured to communicate with the console(s) 28 and/or a server (e.g., a remote server or local server) over one or both of a local area network (LAN) and/or a wide area network (WAN) to receive data (e.g., live data, historical data, etc.) from the console(s) 28 and/or one or more servers. In some examples and as depicted in FIG. 7, one or more second user interfaces 56 may communicate with one or more consoles 28 over a LAN 61 and one or more second user interfaces 56 may communicate with the one or more consoles 28 over a WAN 63, but other suitable network connections are contemplated. In some cases, the LAN 61 may be a network local to a building or company system and may include wired and/or wireless network connections between computing devices of the network. The WAN 63 may be a network that may have or include a cloud computing connection (e.g., a web server connection) and/or other suitable connection for connecting with devices that may or may be part of the LAN 61. The WAN 63 may include wired and/or wireless network connections. When a web server or other server is utilized, the server may be configured to translate data (e.g., a data stream) into a format (e.g., a web page, a web socket, app data, etc.) that facilitates allowing users to consume the data via the remote display 58.

In some cases, the computing devices that may include or may be in communication with the remote display(s) 58 may include one or more application programming interfaces (APIs) configured to facilitate communication (e.g., one-way and/or two-way communication) between the second user interface 56 and the console 28. When included, the API may facilitate the computing device of the second user interface 56 connecting to and/or communicating with two or more consoles 28 at a time.

In some cases, the API may facilitate displaying the historical data and/or archived data on the remote display. For example, the API may be specific to a company that produces the console 28, specific to one or more consoles 28 from which information is to be displayed on the second user interface(s) 56, specific to certain data from one or more consoles 28 (e.g., allow historical information, but prohibit certain bibliographic patient information from the console 28), and/or specific to one or more other features related to the console 28. In some examples, one or more API keys may be utilized to restrict access at the second user interface(s) 56, where the API key may be utilized to provide access at the second user interface(s) 56 to information from a particular company's products, to information from one or more specific consoles 28 or related devices, to information specific to certain data from one or more consoles 28 or related devices, and/or to information specific to one or more other features related to the console 28.

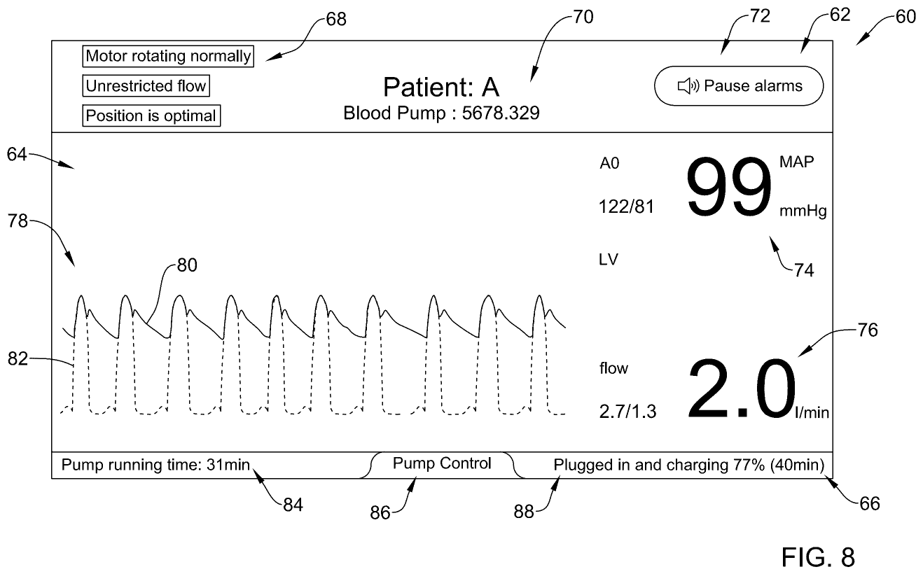

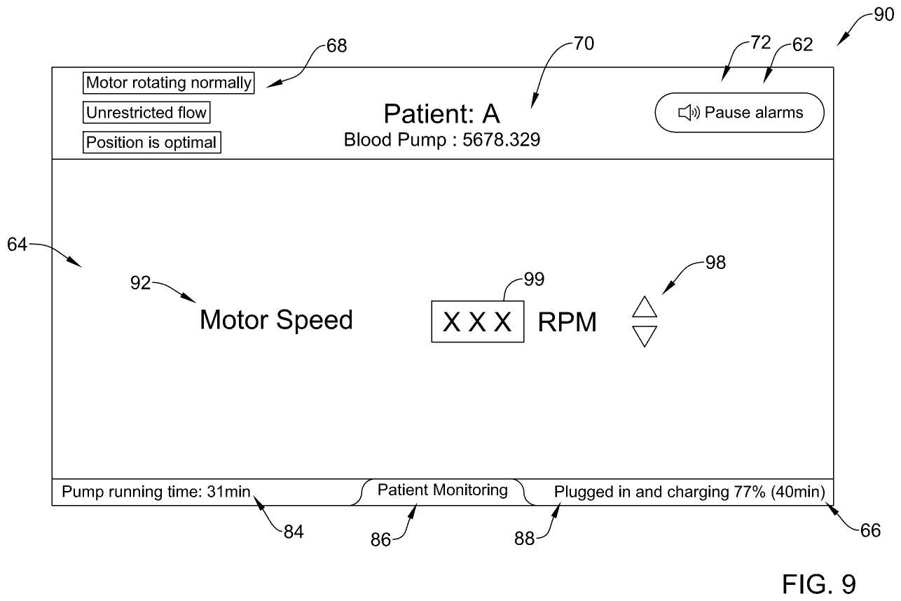

FIGS. 8 and 9 schematically depict illustrative configurations of screens configured for display on the console display 54 of the first user interface 52 and/or the remote display 58 of the second user interface 56. FIG. 8 schematically depicts an illustrative configuration of a patient parameter live screen 60. FIG. 9 schematically depicts an illustrative control screen 90 for controlling operation of the blood pump 24. The patient parameter live screen 60 and the control screen 90 depicted in FIGS. 8 and 9 may be configured for use on a touchscreen display and/or a non-touchscreen display. In some example configurations, the patient parameter live screen 60 and/or the control screen 90 may include a header 62, a body, 64, and a footer 66, but other suitable configurations are contemplated were the header 62, the body 64, and/or the footer 66 are omitted or the information thereat may be located elsewhere or omitted.

The illustrative patient parameter live screen 60 depicted in FIG. 8 may be configured to display relevant current or live patient and/or blood pump data and/or other information in a preconfigured, fixed layout with a format configured to be readable on the console display 54 of the first user interface 52. Although not required, some of the data and/or information provided on the console display 54 may be provided with fixed segments that have a permanent location on the screen, while other data may be provided with dynamic segments, which may facilitate mitigating electrical power usage and/or processing power requirements for the console 28 in a manner that allows for reducing a size of the console 28. For example, a type of data (e.g., MAP, mmHg, l/min, etc.) may be provided in fixed segments, while the values of the data may be provided in dynamic segments.

The header 62 of the patient parameter live screen 60 may include any suitable information related to the patient and/or operation of the blood pump 24. In some examples, the header 62 of the patient parameter live screen 60 may include, among other information or data, blood pump status information 68, patient and/or blood pump identification information 70, one or more alarm buttons (e.g., physical or virtual buttons) 72, and/or other information and/or user interface features.

The blood pump status information 68 may include information from or for the blood pump 24 (e.g., from the motor of the blood pump 24, from sensors of the blood pump 24, manual entries based on imaging of the blood pump 24, etc.). Example suitable status information 68 for the blood pump 24 includes, but is not limited to, one or more indications of a motor rotation status (e.g., “MOTOR ROTATING NORMALLY” or other suitable status indication), one or more flow through the blood pump 24 (e.g., “UNRESTRICTED FLOW” or other suitable status indication), one or more indications of a positioning of the blood pump 24 (e.g., “POSITION IS OPTIMAL” or other suitable status indication), and/or other suitable status information 68 for the blood pump 24.

The patient and/or blood pump identification information 70 may include information that may be used to identify the patient and/or the blood pump 24 coupled with the console 28. Example suitable patient and/or blood pump identification information includes, but is not limited to, one or more patient identification indicators (e.g., a patient name, such as “PATIENT: A” (e.g., where A is an illustrative patient name or a placeholder for a patient name), a patient identification number, and/or other suitable information related to the patient), one or more blood pump identification indicators (e.g., a blood pump serial number (e.g., 5678.329), blood pump model number, and/or other suitable information related to the blood pump 24), and/or other suitable identification information 70 for the patient and/or the blood pump 24.

The one or more blood pump alarm buttons 72 may be virtual and/or physical and may allow a user to interact with an alarm system of the blood pump 24 and/or the console 28. Example suitable blood pump alarm buttons 72 may include, but are not limited to, a “PAUSE ALARMS” button that if selected may pause an alarm for a predetermined set of time or until a further occurrence of a parameter value that triggered the alarm, a “STOP ALARMS” button configured to stop an alarm if selected, an “INITIATE ALARMS” button, and/or other blood pump alarm buttons 72 configured to facilitate control of the alarm system. In some cases, selection of a blood pump alarm button 72 may be selection of a general blood pump alarm button 72 for a plurality of alarms and may pop-up further blood pump alarm buttons that may be individually selected and relate to individual alarms of the blood pump 24.

The body 64 of the patient parameter live screen 60 may include any suitable information related to the patient and/or operation of the blood pump 24. In some examples, the body 64 of the patient parameter live screen 60 may include a patient pressure portion 74, a blood flow portion 76, a graph portion 78 (e.g., depicting waveform data), and/or other suitable portions.

The patient pressure portion 74 may be configured to display values of one or more pressure parameters within the patient. In some examples, the patient pressure portion 74 may include one or more values of mean arterial pressure (MAP, which may be 99 mmHg, as depicted in FIG. 8), a blood pressure, an aortic pressure (AO, where 122 is a maximum aortic pressure and 81 is a minimum aortic pressure, as depicted in FIG. 8), a left ventricular pressure (LV), and/or other suitable pressure or pressure related values. The pressure parameter values may be updated at any suitable interval including, but not limited to, every beat of the patient's heart, every second, every minute, every five minutes, every thirty minutes, and/or over one or more other suitable periods of time

The blood flow portion 76 may be configured to display values of one or more blood flow parameters within the patient. In some examples, the blood flow portion 76 may include one or more values updated every beat of a patient's heart, which may include a blood flow rate through the blood pump 24 (e.g., 2.0 l/min, as depicted in FIG. 8, or other suitable value), a maximum blood flow reading (e.g., 2.7 l/min, as depicted in FIG. 8 for example, or other suitable value), a minimum blood flow reading (e.g., 1.3 l/min, as depicted in FIG. 8 for example, or other suitable value), and/or other suitable blood flow rate related data. Alternatively or additionally, the one or more blood flow parameter values may be updated at other suitable intervals including, but not limited to, every second, evert minute, every five minutes, every 30 minutes, and/or over one or more other suitable periods of time.

Although not required, one or more pressure related values and/or blood flow related values may be highlighted (e.g., bolded, underlines, enlarged, color-coded, etc.) such that users are able to easily observe and recognize that value. In some examples, a value may be highlighted in response to selecting the value on the console display 54.

The graph portion 78 may be configured to display graphs of one or more values over time or relative to another parameter (e.g., waveform data). In some examples, the graph portion 78 may display a graph of ventricular pressure (e.g., left ventricular pressure or other suitable ventricular pressure), aortic pressure, MAP, blood flow rate, and/or one or more graphs of other suitable parameters. For example and as depicted in FIG. 8, the graph portion 78 may include an aortic pressure over time graph 80 (e.g., shown in a solid line) and a ventricular pressure over time (e.g., left ventricular pressure over time) graph 82 (e.g., shown in a broken line), but other suitable configurations of the graph portion 78 are contemplated. Although the graph of FIG. 8 does not include units or ranges of units, the graph portion 78 of the patient parameter live screen 60 may include units and/or ranges of units, as desired.

In some examples, the graph portion 78 may be dynamic based on a selection of one or more portions of the patient parameter live screen 60 (e.g., when configured display on the remote display 58). For example, a subject may select the patient pressure portion 74 to display graph(s) of one or more values of or related to patient pressures and a subject may select the blood flow portion 76 to display graph(s) of one or more values of or related to blood flow through the blood pump 24.

The footer 66 of the patient parameter live screen 60 may include any suitable information related to the patient and/or operation of the blood pump 24, along with alternative screen options. In some examples, the footer 66 of the patient parameter live screen 60 may include one or more pump runtime indications 84 indicating a length of time the pump has been running (e.g., 31 minutes, as depicted in FIG. 8), one or more alternative screen tabs 86 that may be selected to display an alternative screen (e.g., a tab labeled “PUMP CONTROL”, as depicted in FIG. 8, and/or other suitable tabs), one or more battery level indications 88 (e.g., a percent charged, such as 77% as depicted in FIG. 8, an amount of time a battery would last while providing energy to the blood pump 24 and the console 28 on its own, an indication the battery is being charged, such as “PLUGGED IN AND CHARGING”, as depicted in FIG. 8, and/or other suitable battery level indications), and/or other suitable information and/or tabs. In some examples, when a user selects an alternative screen tab 86 to display a screen related to the selected tab on the console display 54, which may result in the previously displayed screen may have an alternative screen tab 86 in the footer 66, but other suitable configurations are contemplated.

The illustrative control screen 90 depicted in FIG. 9 may be configured to display options for a user to adjust control settings of one or more parameters related to operation of the blood pump 24. Example suitable blood pump related parameters for which a user may set setpoints from the control screen 90 include motor speed 92 (e.g., in RPMs), pump flow rate or blood flow rate through the pump (e.g., liters/minute (l/m)),, and/or other suitable parameters.

A user may be able to adjust setpoints for the blood pump related parameter(s) in any suitable manner. In some examples, a user may select one or more adjustment features 98 (e.g., up and/or down arrows, a plus-sign, a minus-sign, a scroll bar, and/or other suitable adjustable buttons) associated with a parameter to adjust a value for a setpoint of that parameter that is displayed proximate to a parameter value box 99 (e.g., where XXX in the parameter value boxes 99 is a value or placeholder for a value of the parameter). Alternatively or additionally, a user may be able to adjust a value of a setpoint for a blood pump related parameter by selecting the parameter or component associated with the parameter (e.g., the parameter value box 99, the adjustment feature 98, etc.) and adjusting a user input device in a manner that adjusts the setpoint. Other suitable configurations for adjusting setpoints for operating the blood pump 24 are contemplated.

In some examples, the header 62 and the footer 66 of the control screen 90 may be the same as or similar to the header 62 and the footer 66 of the patient parameter live screen 60, but other suitable configurations are contemplated. In one example, the header 62 and the footer 66 in the control screen 90 may be the same as the header 62 and the footer 66 in the patient parameter live screen 60, except the alternative screen tab 86 may be for the patient parameter live screen 60 (e.g., the alternative screen tab 86 may be labeled “PATIENT MONITORING” to indicate the patient parameter live screen 60 may be displayed if the alternative screen tab 86 is selected).

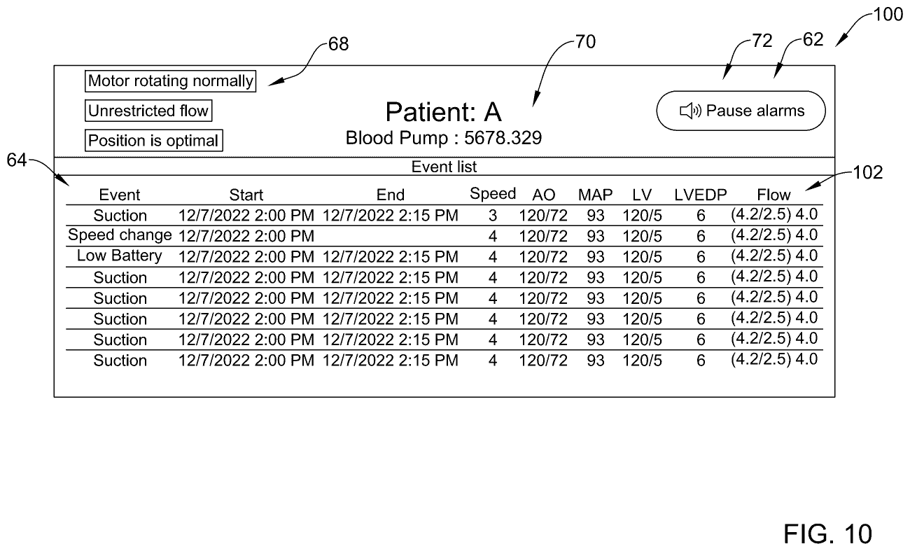

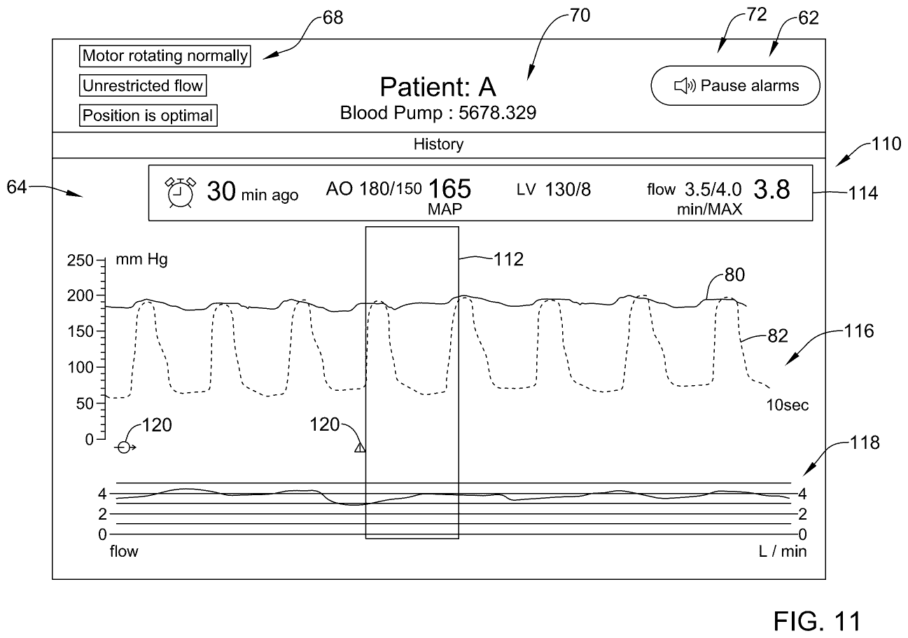

FIGS. 10 and 11 schematically depict illustrative configurations of screens for display on the remote display 58 of the second user interface 56. FIG. 10 schematically depicts an illustrative configuration a patient and/or blood pump event list screen 100. FIG. 11 schematically depicts an illustrative parameter history screen 110. The event list screen 100 and the parameter history screen 110 depicted in FIGS. 10 and 11, respectively, may be configured for use on a touchscreen display and/or a non-touchscreen display. In some example configurations, the event list screen 100 and/or the parameter history screen 110 may include the header 62, the body, 64, and/or the footer 66, but other suitable configurations are contemplated were the header 62, the body 64, and/or the footer 66 are omitted or the information thereat may be located elsewhere or omitted. As depicted in FIGS. 10 and 11, the event list screen 100 and the parameter history screen 110 may include the header 62 and the body 64, where the header 62 may be the same as or similar to the configuration of the header 62 depicted in FIGS. 8 and 9, but other suitable configurations are contemplated.

The illustrative patient event list screen 100 depicted in FIG. 10 may be configured to display events (e.g., alarms and/or other suitable events) that have occurred during the use of the blood pump 24 with the patient and blood pump 24 noted in the header 62, where the events list shows all of or a portion of the events that have occurred since a first initiation of the blood pump 24 within the patient since another instance of time. Although not depicted in FIG. 10, the event list screen 100 may include arrows and/or a scroll bar to view different events over time. Further, events may be sortable by selecting a title 102 for data in the event list screen 100 (e.g., a column title such as “Event”, “Start”, “End”, “Speed”, “AO”, “MAP”, “LV”, “LVEDP”, “Flow”, and/or other suitable titles).

An event may be noted when a value of one or more monitored parameters related to operation of the blood pump 24 reaches or goes beyond a threshold. In one example, a low battery event may be initiated when there is fifteen minutes of battery power left and/or battery power reaches or exceeds one or more other suitable threshold values. Example suitable events include, but are not limited to a suction event, a speed change event, a low battery event, a dead battery event, a high battery event, a battery communication event, a battery critical event, a sudden pressure change event, a motor decoupling event, a motor no commutation event, a motor high current event, a low blood flow event, a no blood flow alert, a low MAP event, a critical MAP alert, a poor blood pump positioning event, a blood pump out of positioning event, and/or other suitable events. When an event occurs, various information at the time of or related to the event may be noted and/or saved in the event list screen 100 including, but not limited to, an event type (e.g., “SUCTION”, “SPEED CHANGE”, “LOW BATTERY”, etc.), an event start time, an event end time, if any, a speed of the blood pump 24, an aortic pressure (AO, where maximum (120)/minimum (72) AO pressure is depicted in FIG. 8), a MAP, left ventricular pressure (LV, where maximum (120)/minimum (5) LV pressure is depicted in FIG. 8), a left ventricular end-diastolic pressure (LVEDP), a flow rate of blood through the blood pump 24 (where maximum (4.2)/minimum (2.5) and mean (4.0) are depicted in FIG. 8), and/or other suitable information.

In some cases, a portion of the patient event list screen 100 may be associated with an event (e.g., a row or a portion of a row associated with data of an event) may be a selectable element associated with data received from the console 28. When the selectable element is selected by a user, additional information related to the event or data associated with the specific selectable element may be displayed on the remote display 58. In some examples, selection, by a user, of a selectable element associated with an event on the patient event list screen 100 may result in highlighting data at the time of the selected event in parameter history screen 110 (e.g., as depicted in FIG. 11 and discussed below) and if the parameter history screen 110 is not displayed with the patient event list screen 100 on the remote display 58, may cause the parameter history screen 110 that includes a time period associated with the event or selectable element to be displayed on the remote display 58.

The illustrative parameter history screen 110 depicted in FIG. 11 may be configured to display, in the body 64, data and/or information related to one or more time instances since operation of the blood pump 24 was initiated internal to the patient, where the blood pump 24 and/or the patient are noted in the header 62. The parameter history screen 110 may include one or more graphs of data (e.g., waveform data) related to patient parameters and/or blood pump parameters and a dynamic data portion 114, where the dynamic data portion 114 may display data associated with a location of a slider bar 112 (e.g., an adjustable date and/or time indicator) along the one or more graphs.

The one or more graphs may display information and/or data related to one or more parameters associated with the patient and/or operation of the blood pump 24. A first graph 116 may depict the aortic pressure (mmHg) graph 80 and the left ventricular pressure (mmHg) graph 82 over time and a second graph 118 may depict a flow rate (l/min) of blood through the blood pump 24 over time, where a timeline (e.g., where time may be the horizontal axis) of the pressures of the patient and the flow rate of the blood through the blood pump 24 are the same or otherwise aligned.

The slide bar 112 may extend through (e.g., vertically through) the first graph 116 and the second graph 118, and the slide bar 112 may be adjusted (e.g., adjusted horizontally) to highlight conditions related to operation of the blood pump 24 at different times. In some examples, as the slide bar is adjusted along the first graph 116 and/or the second graph 118, the values of parameters in the dynamic data portion 114 may adjust to display values associated with a time at which the slide bar 112 is located along the first graph 116 and/or the second graph 118.

The slide bar 112 may be adjusted in any suitable manner. In some examples, the slide bar 112 may be adjusted by selecting the slide bar 112 and adjusting the slide bar 112 along one or both of the first graph 116 and the second graph 118. Alternatively or additionally, the slide bar 112 may be adjusted by selecting a location along one or both of the first graph 116 and the second graph 118, which may automatically result in the slide bar 112 being located at or centered on the location selected.

The data depicted in the dynamic data portion 114 may be associated with a time aligned with a center of the slide bar 112, but other configurations are contemplated. As depicted in FIG. 11, the slide bar 112 is associated with a time of thirty (30) minutes in the past, at which the patient's blood pressure was 180/150, the patient's MAP was 165 mmHg, the patient's left ventricular pressure was 130/8, a maximum flow rate of blood through the blood pump 24 during a range of time represented by the slide bar 112 (e.g., represented by a width of the slide bar 112, which is not drawn to scale) is 4.0 l/min, a minimum flow rate of blood through the blood pump 24 during the range of time represented by the slide bar 112 is 3.5 l/min, and a flow rate of blood through the blood pump 24 at a time associated with the center of the slide bar is 3.8 l/min. Additional or alternative data may be displayed at the dynamic data portion 114 of the parameter history screen 110.

The graph portion of the parameter history screen 110 may include one or more event indicators 120 (e.g., on an alarm indication) and/or other suitable indicators, as depicted in FIG. 11. In some examples, an event indicator 120 may indicate an event occurred at a time (e.g., a particular time, a heartbeat, etc.) associated with a location of the event indicator 120 in the graph portion of the parameter history screen 110. Selection of the event indicator 120 may result in adjustment of the slide bar 112 to the location of the event indicator 120, adjustment of the data depicted in the dynamic data portion 114 to values associated with an event associated with the event indicator 120, a pop-up screen providing further data related to the event associated with the event indicator 120, and/or other suitable adjustments and/or information being displayed. Different shaped event indicators may represent different events.

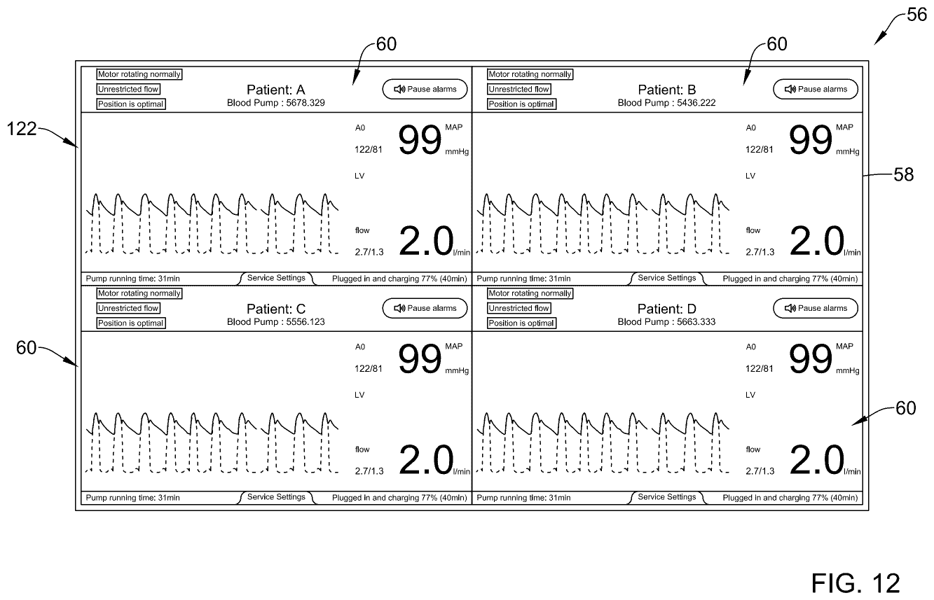

FIGS. 12-15 depict different arrangements of various screens associated with patients that may be displayed on the remote display 58 of the second user interface 56. Although the data of the screens depicted in FIGS. 12-15 for different patients (e.g., patients A-B) may be the same, it is contemplated that different patients will have different data displayed on the various screens.

The various screens associated patients may be presented on the remote display 58 simultaneously as panes (e.g., patient panes). In some examples, the panes may be automatically adjusted as additional and/or alternative panes are presented on the remote display. Alternatively or additionally, the panes may be manually adjustable in size and/or location on the remote display 58 relative to one another. Further, panes may be added to and/or removed from the remote display 58 by a user. In some examples, a user may drag and drop screens for one or more patients into or out of a screen of the remote display 58, a user may search for a patient and add one or more screens for the searched-for patient into a screen of the remote display 58 by selecting the searched-for patient, and/or the panes may be added to and/or removed from the remote display 58 in one or more other suitable manners.

FIG. 12 schematically depicts a patient view screen 122 on the remote display 58, where patient parameter live screens 60 are displayed or otherwise provided for a plurality of patients (e.g., for four patients, as depicted in FIG. 12, where each patient parameter live screen 60 is a pane in the patient view screen 122). In some examples, the patient parameter live screens 60, or portions thereof, in the patient view screen 122 may have selectable elements associated with data. Selection of one or more of the patient parameter live screens 60 or portions thereof in the patient view screen 122 may result in further details (e.g., additional and/or alternative screens and/or further details presented in one or more other suitable manners) related to the selected patient parameter live screen 60 or portion thereof being displayed in the remote display 58. In some examples, when a patient parameter live screen 60 is selected from the patient view screen 122, the event list screen 100 and/or the parameter history screen 110 may be displayed on the remote display 58 without the selected patient parameter live screen 60 being displayed.

Patient parameter live screens 60 may be displayed on the remote display 58 for any suitable number of patients (e.g., one patient, two patients, three patients, four patients, etc.) When one or more patient parameter live screens 60 are provided for more patients than can be displayed on the remote display 58 at one time (e.g., simultaneously), the patient view screen 122 may be scrollable in any suitable manner, searchable in any suitable manner, and/or otherwise adjustable to view patient parameter live screens 60 not otherwise shown on the remote display 58.

In some examples, locations of the patient parameter live screens 60 displayed on the remote display 58 may be adjusted. For example, the locations of the patient parameter live screens 60 may be manually adjusted (e.g., via a user selecting one or more live patient parameter screens and moving (e.g., dragging, etc.) the selected screens to a different location in the remote display 58, etc.) and/or automatically adjusted (e.g., the patient parameter live screens 60 may be automatically adjusted to a top of the remote display 58 based on one or more alarms, a time since last alarm, a time since an associated blood pump 24 was initiated, and/or other suitable priority settings, etc.).

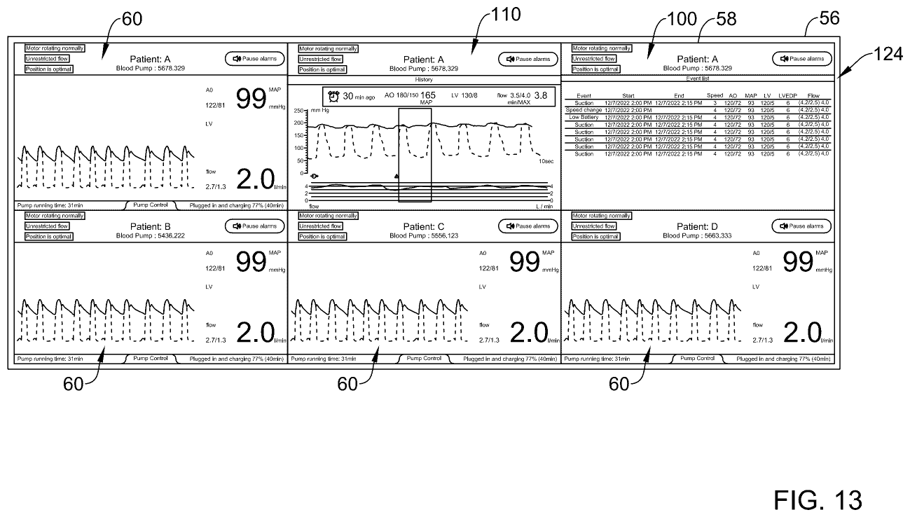

FIG. 13 schematically depicts an illustrative configuration of a detailed patient view screen 124 (e.g., a detailed view for a selected patient) displayed in the remote display 58 of the second user interface 56, which may be initiated by selecting a patient from the patient view screen 122 (e.g., selecting a patient parameter live screen 60 associated with a patient). In the configuration of the detailed patient view screen 124 depicted in FIG. 13, the remote display 58 may adjust the patient parameter live screens 60 of the patient view screen 122 such that the selected patient parameter live screen 60 (e.g., the patient parameter live screen 60 of Patient A) may be displayed in a top row next to the parameter history screen 110 for Patient A and the event list screen 100 for Patient A, while the patient parameter live screens 60 for non-selected patients (e.g., Patient B, Patient C, and Patient D, as depicted in FIG. 13) are depicted below the row of detailed data screens (e.g., screens 60, 100, 110 for Patient A). In some examples, the screens of the selected patient (e.g., Patient A) may be displayed in a larger font or dimension than a font or dimension used for the patient parameter live screens 60 of the non-selected patients (e.g., Patient B, Patient C, and Patient D), however, other suitable configurations are contemplated.

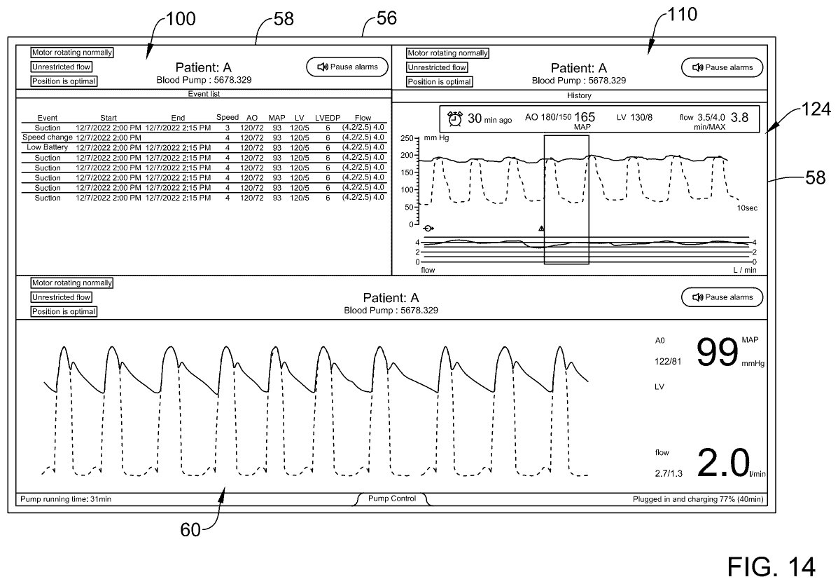

FIG. 14 schematically depicts an illustrative configuration of a detailed patient view screen 124 displayed in the remote display 58 of the second user interface 56, which may be initiated by selecting a patient from the patient view screen 122 (e.g., by selecting a patient parameter live screen 60 associated with a patient). In the configuration of the detailed patient view screen 124 depicted in FIG. 14, the second user interface 56 may display screens associated with the selected patient (e.g., patient A) and screens associated with non-selected patients may be omitted. In one example, the second user interface 56 may display the detailed data screens associated with the selected patient in a top or first row and the patient parameter live screen 60 associated with the selected patient in the bottom or second row. In some examples, the detailed data screens of the selected patient may be displayed in larger font than a font used for the patient parameter live screens 60 of the selected patient. Other suitable configurations of the screens associated with the selected patient are contemplated.

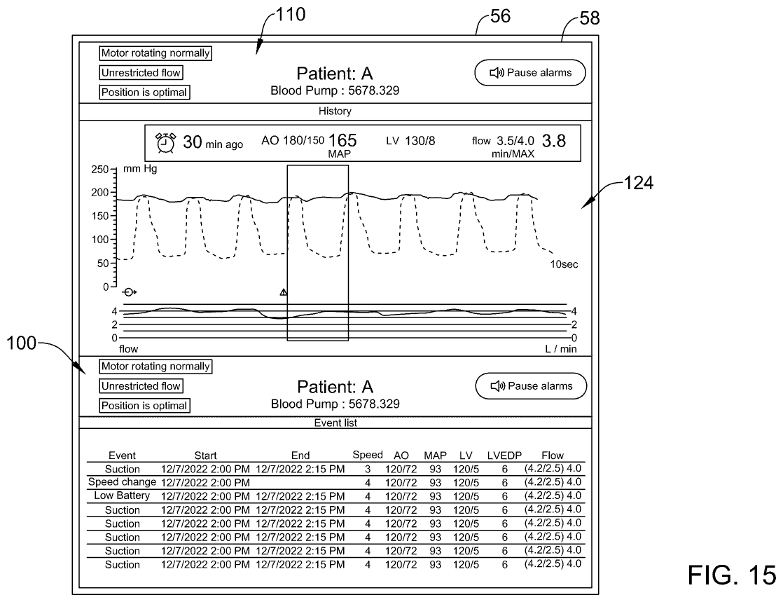

FIG. 15 schematically depicts an illustrative configuration of a detailed patient view screen 124 displayed in the remote display 58 of the second user interface 56, which may be initiated by selecting a patient from the patient view screen 122 (e.g., by selecting a patient parameter live screen 60 associated with a patient). In the configuration of the detailed patient view screen 124 depicted in FIG. 14, the remote display 58 may display detailed data screens associated with the selected patient (e.g., patient A) and the patient parameter live screens 60 (e.g., of the selected and the non-selected patients) may be omitted. In one example, the remote display 58 may display the parameter history screen 110 associated with the selected patient in a top row and the event list screen 100 associated with the selected patient in the bottom row. Other suitable configurations of the screens associated with the selected patient are contemplated.

As discussed, the percutaneous circulatory support system 10 may be utilized in one or more illustrative techniques for displaying information and/or data related to operation of the blood pump 24 to users. In some examples, the console 28 of the percutaneous circulatory support system 10 may receive information and/or data over time from the blood pump 24. The information and/or data received over time may be related to the patient, the blood pump, and/or operation of the blood pump 24 (e.g., operation of the blood pump 24 within the patient). As the information and/or data is received from the blood pump, the information and/or data received over time may be displayed live on the first user interface 52 (e.g., on the console display 54) of the console 28. In some examples, the information and/or data received over time may be displayed in a preconfigured, fixed layout or configuration to a facilitate a portable nature of the console 28 and effectively relaying patient and/or blood pump related information to users of the console 28.

The information and/or data related to operation of the blood pump 24 that is received over time at the console 28 from the blood pump 24 may be communicated to a remote computing device having the second user interface 56. Further, the information and/or data received over time at the console 28 may be saved or otherwise stored on the console 28 and/or communicated or transferred to a server (e.g., a remote or local server) for storage and access by the console 28 and/or the remote computing device(s) for display on the second user interface 56.

The information and/or data communicated to the remote computing device and the second user interface 58 may be displayed on the second user interface 56 (e.g., on the remote display 58). In some cases, the information and/or data communicated to the remote computing device having the second user interface 56 may be communicated to a plurality of remote computing devices having the second user interfaces 56 and displayed on respective remote displays 58 of the second user interfaces 56 (e.g., displayed on remote displays of a first one of the second user interfaces 56, a second one of the second user interfaces 56, a third one of the second user interfaces 56, and so on). The information and/or data may be displayed on the remote display 58 using a patient parameter live screen 60, an event list screen 100, a parameter history screen 110, a patient view screen 122, a detailed patient view screen 124, and/or one or more other suitable screens.

In some examples, the second user interface may receive information and/or data related to operation of a plurality of blood pumps 24 and the received information and/or data may be display in the patient view screen 122 depicting a plurality of patient parameter live screens 60 each associated with a different patient. A user may select a patient parameter live screen 60 (e.g., a selectable element) of the plurality of patient parameter live screens 60 and in response to receiving the selection of the patient parameter live screen 60, one or more detailed views that include data associated with the selected patient parameter live screen 60 may be displayed. Example suitable detailed views include, but are not limited to, the screens depicted in and described with respect to FIGS. 10-15. In one example, an event list screen 100 and/or a parameter history screen 110 may be displayed on the remote display 58 in response to selecting the patient parameter live screen 60, but this is not required.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A circulatory support system comprising:
a blood pump;
a console in communication with the blood pump, the console comprising a controller configured to control operation of the blood pump;
a first user interface configured to display a live view related to operation of the blood pump, wherein the first user interface is part of the console; and
a second user interface configured to display live data and historical data of one or more parameters related to operation of the blood pump, wherein the second user interface is remote from the console.

2. The circulatory support system of claim 1, wherein the first user interface is configured to display a first screen including the live view related to operation of the blood pump and a second screen configured to facilitate a user controlling operation of the blood pump.

3. The circulatory support system of claim 2, wherein the first screen and the second screen have preconfigured fixed layouts and are the only screens that the console is configured to display.

4. The circulatory support system of claim 1, wherein the second user interface is a dynamic user interface configured to adjust between a plurality of screens based on user interaction with a screen of the plurality of screens displayed on the second user interface.

5. The circulatory support system of claim 4, wherein the plurality of screens are configured to display one or more of a live view of one or more patient parameters related to operation of the blood pump, a historical view of one or more patient parameters related to operation of the blood pump, operational settings of the blood pump, and a view of one or more patient parameters related to operation of the blood pump for a plurality of patients.

6. The circulatory support system of claim 1, wherein the second user interface includes one or more selectable elements associated with data received from the console and the one or more selectable elements are configured to be selected to cause the second user interface to display a screen with additional information related to the data received from the console.

7. The circulatory support system of claim 6, wherein the one or more selectable elements is a patient pane on a screen displaying a plurality of patient panes and the screen with additional information related to the data received from the console includes a plurality of adjustable patient panes that include one or more of a live view of one or more patient parameters, historical alarm data, and historical waveform data.

8. The circulatory support system of claim 1, wherein the first user interface includes a first display having a first surface area and the second user interface includes a second display having a second surface area greater than the first surface area.

9. The circulatory support system of claim 1, wherein the second user interface is configured to receive one or both of the live data and the historical data from the console over a local area network.

10. The circulatory support system of claim 1, wherein the second user interface is configured to receive one or both of the live data and the historical data from the console over a wide area network.

11. The circulatory support system of claim 1, wherein the console is configured to store the live data and the historical data of one or more parameters related to operation of the blood pump.

12. The circulatory support system of claim 1, wherein the console is configured to output the live data and the historical data of one or more parameters related to operation of the blood pump to a server in communication with a computing device comprising the second user interface.

13. A console for a circulatory support system, comprising:
a controller configured to control operation of a blood pump of the circulatory support system;
a first display configured to display a live view of one or more parameters related to operation of the blood pump; and
wherein the controller is configured to store and output live data and historical data of one or more parameters related to operation of the blood pump.

14. The console of claim 13, wherein the controller is configured to communicate with the blood pump over a wired connection.

15. The console of claim 13, wherein the controller is configured to output the live data and the historical data of one or more parameters related to operation of the blood pump to a remote user interface over a wired connection.

16. The console of claim 13, wherein the controller is configured to output the live data and the historical data of one or more parameters related to operation of the blood pump to a remote user interface over a wireless connection.

17. A method comprising:
receiving data over time at a console from a blood pump operating within a patient, wherein the data received includes data related to operation of the blood pump within the patient;
displaying live data from the data received over time on a first user interface of the console;
communicating the data received over time from the console to a remote computing device having a second user interface; and
displaying data related to operation of the blood pump on the second user interface.

18. The method of claim 17, further comprising:
storing the data received over time on the console.

19. The method of claim 17, further comprising:
displaying the live data and historical data on a third user interface remote from the first user interface and the second user interface.

20. The method of claim 17, further comprising:
receiving a selection of a selectable element on a screen of the second user interface, wherein the screen is displaying at least some of the data related to operation of the blood pump; and
in response to receiving the selection of the selectable element, displaying a detailed view that includes data associated with the selectable element.
